(19) United States
(12) Patent Application Publication
(10) Pub. No.: US 2007/0026025 A1
(43) Pub. Date: Feb. 1, 2007

(54) TOPICAL OINTMENT AND METHOD FOR MAKING AND USING SAME

(75) Inventor: Stephen M. Mitchell, Brighton, CO (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Aquegel Cosmetics, LLC, Brighton, CO (US)

(21) Appl. No.: 11/462,105

(22) Filed: Aug. 3, 2006

Related U.S. Application Data
(63) Continuation-in-part of application No. 11/104,944, filed on Apr. 12, 2005.

(51) Int. Cl. A61K 9/14; A61K 9/00

(52) U.S. Cl. 424/400; 424/488

(57) ABSTRACT

A topical ointment comprises a base material of plasticized hydrocarbon gel and methylcellulose in which are dispersed a plurality of microbubbles containing liquid. The microbubbles containing liquid are encapsulated by the base material to form microencapsulations which are dispersed in the base material to form a hydrogel. Application of the topical ointment to a body area provides for the moisturizing and the slow delivery of the liquid in the microencapsulations to the applied area. Body heat melts the base material and the contents of the microencapsulations are thereby released.

### CROSS-REFERENCE TO RELATED APPLICATIONS

This application is a continuation-in-part of U.S. patent application Ser. No. 11/104,944, filed Apr. 12, 2005, which is incorporated by reference herein in its entirety.

### FIELD OF THE INVENTION

The present invention relates to a topical ointment and more particularly to a topical ointment which may act as a moisturizer and delivery system of substances to bodily areas upon which it is applied.

### BACKGROUND OF THE INVENTION

A number of methods and apparatus exist for delivering drugs and other pharmaceuticals to parts of the human body. In oral delivery of the drug, the active agent enters the bloodstream by being absorbed in the lining of the stomach. Another drug delivery system is through direct injection via a needle into the bloodstream. Other possible drug delivery systems include the administration of a suppository, endotracheal administration, and eye dropping administration. Disadvantages of the above described methods may include lack of absorption of the drug through stomach lining, the pain experienced from injections, and the inability to deliver measured amounts of the drugs over predetermined periods of time.

Another method of delivering a drug or pharmaceutical is through application of a substance carrying the drugs or pharmaceuticals to a mucosal surface of the body. The drug or other pharmaceutical composition may be mixed with a petroleum-based jelly and the combination topically applied to a mucosal surface, such as those in the nasal passageway. As is documented, the placement of petroleum-based products in the airways, may have certain medical side effects.

### SUMMARY OF THE INVENTION

The invention provides topical ointments that supply, among other features, moisturization upon application to various body surfaces. In situations where the ointment includes additional substances such as medications, the ointment may further provide for the controlled delivery of these substances to surfaces upon which the ointment has been applied. Upon application of the topical ointment to different body areas, the delivery of the additional substances to the surfaces within the area may occur at a slow release rate.

The topical ointment may comprise a base material of plasticized hydrocarbon gel and methylcellulose. A plurality of microbubbles containing liquid is encapsulated by the base material to form microencapsulations that are dispersed in the base material to form a hydrogel. In one particular embodiment, the plasticized hydrocarbon gel may comprise a combination of mineral oil and polyethylene glycol (“PEG”). The microencapsulations may include a quantity of a liquid, such as an aqueous solution or sterile water. Examples of aqueous solutions that may be encapsulated include a solution of sodium chloride (NaCl) in sterile water, and a bacteriostatic solution comprising NaCl, benzyl alcohol and sterile water.

One or more additional substances may be included in the liquid within the microencapsulations. These additional substances may include, but are not limited to, various drugs or pharmaceutical substances such as antibiotics, steroids, aromatic oils, nitroglycerine, painkillers, nicotine and humalog insulin.

In its broadest aspects, the ointment manufacturing process comprises the steps of providing a plasticized hydrocarbon gel; mixing the plasticized hydrocarbon gel with a liquid solution; mixing a first quantity of methylcellulose into the mixture in small incremental amounts; increasing the shear rate of mixing to a level sufficient to form microbubbles on the surface; mixing a second quantity of methylcellulose into the mixture until the microbubbles are incorporated into the mixture and the liquid has moved into the microbubbles to form microencapsulations; and reducing the shear rate of mixing and continuing to mix the mixture for a time sufficient to fully disperse the microencapulations and form a fully bonded hydrogel.

In the manufacturing process for the topical ointment wherein a commercially available plasticized hydrocarbon gel is provided, a quantity of the plasticized hydrocarbon gel and a quantity of a liquid to be incorporated in the ointment may be placed in a mixing device and mixed at a first slow shear rate that is sufficient to thoroughly mix the liquid with the gel. Preferably, the first shear rate is in the range of 45-65 rpm. The mixture may be then mixed at a second shear rate that is sufficient to form a vortex. Preferably, the second shear rate is in the range of 65-98 rpm. A quantity of methylcellulose may then be added to the vortex is several increments, preferably 2-4 substantially equal increments, until about ⅔ of the methylcellulose has been added and the mixture is mixed until a foam of microbubbles appears on the surface layer. The material is then mixed at a higher third shear rate sufficient to form microbubbles of the desired size and the remaining methylcellulose is added. Preferably, the third shear rate is in the range of 98-130 rpm. The mixing process is continued at the higher third rate so as to incorporate all of the microbubbles into the mixture and allow the previously added liquid to move osmotically into the microbubbles. The mixing rate is then reduced to a shear rate, and continued for a period of time, sufficient to fully disperse the microencapsulations in the base material and form a fully bonded hydrogel. Preferably, this last shear rate is in the range of 45-65 rpm.

In the manufacturing process for the topical ointment wherein the plasticized hydrocarbon gel is provided as part of the process, a quantity of PEG may heated until liquefied in a mixing device, e.g. a homogenizer, and then continuously mixed at a low shear rate with a quantity of mineral oil and allowed to cool to a temperature below 35° C., thereby forming the plasticized hydrocarbon gel. Separately, a liquid solution to be incorporated into the ointment may be prepared and then added to the cooled plasticized hydrocarbon gel while the low shear mixing is continued. Once fully mixed, methylcellulose may be added in small increments to the mixture until about ⅓ of the methylcellulose is remaining. The speed of the mixing may then be increased to a high shear rate and mixing continued until a foam of microbubbles appears on the surface, at which time the remaining ⅓ of the methylcellulose may be added. The high shear mixing may be continued until all of the microbubbles are incorporated in the mixture. The mixing speed may then be reduced to a low shear rate and continued until the temperature of the mixture drops to room temperature.

After mixing, the hydrogel may be allowed to sit for 1-78 hours so as to let the microencapsulations settle and reduce in size, thereby increasing the number microencapsulations per volume of ointment. The ointment may then be placed in suitable containers, e.g. bottles, and refrigerated for 2 hours to 4 days to allow the product to thicken to the desired viscosity. The containers may then be returned to room temperature and will remain relatively stable.

Additional substances may be added to the mixture, preferably with the bacteriostatic saline solution. Portions of the liquid containing the additional substance may be added to the base material during the mixing process until the entire amount of liquid is encapsulated.

In use, the topical ointment may be applied to various body surfaces. When the ointment comes in contact with the body surface, body heat; e.g. in the range of 90°-105° F., may act to dissolve the base material providing for the release to the surface of the aqueous solution and other substances encapsulated therein. The rate the base material dissolves may be related to the proximity of that portion of the ointment to the body surface as well as other environmental factors.

As the base material dissolves, the materials encapsulated within the base material are delivered to the body surface. This continuous exposure acts to hydrate the affected area as well as provide exposure to the additional substances. Through this exposure medications may be absorbed into the blood stream.

In one specific aspect, the ointment formulation may include a plasticized hydrocarbon gel comprising PEG and mineral oil, methycellulose and bacteriostatic normal saline (e.g. a solution of NaCl and benzyl alcohol). Other bacteriostatic components and buffered normal saline may also be added to this base. The normal saline may be encapsulated in microencapsulations within the base material to create a safe, non-petroleum, time released gel. Further, microencapsulation of the saline provides a time release lasting approximately 8 to 12 hours with a single application. The ointment may also be pH balanced to the nasal mucosa.

### BRIEF DESCRIPTION OF THE DRAWING

FIG. 1 is a partial cross sectional view of an ointment according to the invention.

### DESCRIPTION OF THE SPECIFIC EMBODIMENTS

The topical ointment of the present invention may be applied to various parts of the human body, and through exposure to body heat, may provide delivery of moisturizing agents (hydration), as well as other substances. In one embodiment, the topical ointment comprises a hydrogel wherein gas microbubbles containing a liquid, e.g. sterile water or aqueous solution, are encapsulated in a base material comprising a plasticized hydrocarbon gel and methylcellulose. The gas microbubbles containing liquid are herein referred to as “microencapsulations”. Also included may be additional substances such as medications or aromatic oils, which when encapsulated within the topical ointment provide for a slow release of the substance to a body surface.

As used herein, the term “hydrogel” refers to a reversible hydrocolloid in which the base material forms the external or dispersion phase, and microencapsulations form the internal or dispersed phase. The topical ointment of the present invention is a “reversible hydrocolloid” because it is composed of a base material whose physical state is an elastic gel at ambient temperatures, but becomes a liquid upon heating, e.g. by body heat.

As mentioned, the topical ointment of the present invention includes a base material comprising a combination of a plasticized hydrocarbon gel, i.e. a material that may be both plasticized and gelatinized, and methycellulose. For example, the base material may typically comprise approximately 90-97% plasticized hydrocarbon gel and 3-10% methycellulose. In one embodiment, the base material may comprise 802-968 gms of a plasticized hydrocarbon gel (e.g. a gel comprising 95% mineral oil and 5% PEG) and 38-79 gms of methylcellulose.

Examples of plasticized hydrocarbon gels that may be used include those gels constructed of mineral oil and a polyalkylene alcohol; e.g., polyethylene glycol (“PEG”). Commercially available plasticized hydrocarbon gels include Plastibase®, as well as Eucerin™, Aquaphor™, A&D Ointment™, Noxema™, petrolatum, Nivia™, Vaseline™, Aveeno™ cream or ointment, bees wax, paraffin, Lanolin™, emulsifying ointments or creams, hydrophilic creams or ointments, and the like. One exemplary base material is a plasticized hydrocarbon gel that comprises about 1-9%, and more preferably from about 4-6%, of PEG and about 91-99%, and more preferably from about 94-96%, of mineral oil.

The PEG preferably has a molecular weight in the range from about 1295 to about 1315, and may comprise a combination of different molecular weights. For example, the PEG may comprise about 20 percent of a low molecular weight PEG and about 80 percent of a high molecular weight PEG. As another example, the PEG may comprise about 50 percent of a low molecular weight PEG and about 50 percent of a high molecular weight PEG. Further, it will be appreciated that other combinations are possible that are able to produce a PEG in the molecular weight range of about 1295 to about 1315. Such a polymer is soluble in mineral oil above about 95° Fahrenheit (35° Celsius), which is close to its melting point. When the solution is cooled below 95° Fahrenheit, the polymer precipitates and causes gelatinization.

The mineral oil may be a high-grade pharmaceutical mineral oil having a specific gravity that allows it to bind under complex gradients, i.e., heat and shear, with a low molecular weight PEG. The mineral oil and PEG form a complex matrix of alkylene bonding. Such a formulation results in essentially no residual mineral oil, as it becomes part of the matrix and is incorporated into the complex bonding of the carbons. This creates an irreversible compound which is gelatinized and plasticized. The chemical moiety does not have free mineral oil in its finished state. As such, it does not revert back to its mineral oil form because it has been plasticized.

Methylcellulose (CAS # 9004-67-5), also known as cellulose methyl ether or modified cellulose, is a commercially available material manufactured by the methylation of natural cellulose. Preferred methycellulose has a viscosity (2 gms in 100 ml of solution) of 3,000-5600 cps, preferably about 4000 cps. This viscosity causes the microencapsulations to obtain the preferred small size and makes the final product more stable. Sufficient methycellulose should be present in the ointment formation to accelerate the mixing of the base material during formation of the topical ointment and to prevent the separation of water from the hydrogel after it is formed. The methylcellulose also aids in the formation of the foam of microbubbles caused by the induction of the liquid at high shear rates into the base material.

As stated above, the ointment of the present invention comprises a hydrogel wherein a plurality of microencapsulations is dispersed in the base material. The microencapsulations comprise microbubbles that are at least partially filled with a liquid and are encapsulated by the base material; therefore, the contained liquid is slowly released when the base material melts when exposed to body heat during contact with a body surface. The microencapsulations may have a size in the range from about 10 microns to about 1500 microns, and more preferably from about 25 microns to about 50 microns. The base material may have a viscosity in the range from about 10,000 to 250,000 cps; provided, however that the mixture is thixotropic and the viscosity will decrease when agitated. In another aspect, the base material may have a pH of about 5.7. Depending on whether the base material includes any additives, the base material may have a pH in the range from about 4.0 to about 6.8.

A variety of liquids may be contained within the microencapsulations. For example, one liquid that may be encapsulated in the topical ointment is an aqueous solution. This aqueous solution may include other compounds to be delivered by the ointment upon application to a body surface. Other and/or additional substances that may be included in the microencapsulations include a large variety of drugs, oils, dietary supplements and other medications. These substances may be nontoxic, water soluble or miscible materials that are capable of being absorbed through the skin.

Examples of other substances that may be encapsulated in the topical ointment are antibiotics; e.g. Gentamycin, Tetracycline, Erythromycin, and Cephalexin; and steroids; e.g. Prednisone, Dexamethasone, and Prednisolone.

Other substances that may be encapsulated in the base material are aromatic oils. These oils may be employed in treatments such as aromatherapy. The oils may include: Eucalyptus Oil, Lavender Oil, Rosemary Oil, Pine Needle Oil, Tea Tree Oil, Wintergreen Oil, Peppermint Oil, Spearmint Oil, Camphor Oil, Sage Oil, Jojoba Oil, Cinnamon Oil, Anise Oil, Lemon Oil, Lime Oil, Orange Oil, Clove Oil, Almond Oil, White Pine Oil, Cardamon Oil and Cedar Leaf oil. A preferred formulation for aromatherapy comprises about 27-37 drops of Lavender Oil per 908 gms of base material. This formulation has also been shown to aid in sleep and prevent migraine headaches when applied nasally, since lavender is a vasoconstrictor. In addition, this formulation aids in diaper rash by supplying the needed moisture and lavender to eradicate the condition. Another embodiment comprises about 42-55 drops of Eucalyptus Oil per 908 gms of base material. This embodiment may be applied to stop a congested nasal passage, since Eucalyptus Oil is also a vasoconstrictor. The hydrogel adds the needed moisture.

Still other substances that may be encapsulated within the base material are dietary supplements. These supplements may include: various vitamins, iron, potassium, calcium, potassium, magnesium, copper, zinc and the like. In one embodiment, nasal application of an ointment of the present invention containing encapsulated zinc gluconate, e.g. at a concentration of about 1.06 mg/gm, may shorten the duration of the common cold and prevent invasion of airborne viruses, due to the viral shedding of the zinc gluconate and the protective shield formed over the nasal mucosa by the ointment.

Further examples of other substances that may be encapsulated include various medications such as humalog insulin, anti-nausea medication (e.g. Prochloroperazine or Promethazine), smoking prevention medication (e.g. nicotine resin), painkilling medication (e.g. codeine, hyrocodone), nitro-glycerin, and the like.

One type of liquid that may be contained in a microencapsulation of the ointment is sterile water or an aqueous solution, such as a saline solution. Such an aqueous solution may be added in an amount based on weight to volume. For example, for about 800-1047 grams of the total base material (plasticized hydrocarbon gel and methylcellulose), about 500-1131 mL of an aqueous solution may be added. One particular type of aqueous solution that may be added comprises a combination of 0.9% sodium chloride (saline) solution with benzyl alcohol, e.g. about 1.0-11.0 mL of benzyl alcohol to about 500-1120 mL of 0.9% saline solution. The saline solution may be added to the base material separately from the benzyl alcohol or the benzyl alcohol may be combined with the saline solution before addition to the base material. This combination creates a bacteriostatic effect as it is released onto tissue. For instance, when placed in the nasal mucosa, the nasal passage is kept generally sterile as the solution is released onto the nasal mucosa. Further, the aqueous solution also acts as a moisturizer to keep the nasal mucosa moist. For other areas of the body, the moisture helps to reduce healing times and prevent infection. In other embodiments, the added liquid may comprise benzyl alcohol, alone or in combination with saline, mineral oil, propylene glycol, water or the like.

In one preferred embodiment, a typical batch of ointment may comprise 802-968 gms of plasticized hydrocarbon gel; 38-79 gms of methycellulose 4000 CPS; 500-1120 mL of 0.9% sodium chloride (saline) solution; and 1.0-11.0 mL of benzyl alcohol.

The ointment of the invention may be prepared by continuous or batch processes. As in preparing conventional emulsions, shear forces may be applied to the components by use of mixers, blenders, homogenizers, mills, impingement surfaces, ultra-sound, shaking or vibration.

The plasticized hydrocarbon gel may comprise a variety of plasticized and gelatinized materials, and as previously described may be purchased commercially. Alternatively, the plasticized hydrocarbon gel may be formed during the preparation of the ointment

Generally, the ointment manufacturing process comprises the steps of providing a plasticized hydrocarbon gel; mixing the plasticized hydrocarbon gel with a liquid solution; mixing a first quantity of methylcellulose into the mixture in small incremental amounts; increasing the shear rate of mixing to a level sufficient to form microbubbles on the surface; mixing a second quantity of methylcellulose into the mixture until the microbubbles are incorporated into the mixture and the liquid has moved into the microbubbles to form microencapsulations; and reducing the shear rate of mixing and continuing to mix the mixture for a time sufficient to fully disperse the microencapulations and form a fully bonded hydrogel.

In one embodiment of a process for forming the ointment of the present invention, a quantity of commercially available plasticized hydrocarbon gel may be placed in a mixing device and a whipping or mixing process using a first, low shear rate; e.g. in the range of 45-65 rpm, may then performed on the plasticized hydrocarbon gel. A liquid, such as an aqueous solution and/or any of the additional substances described above, may then be mixed with the plasticized hydrocarbon gel at the first low shear rate. After all of the desired liquid has been added, the shear rate may be increased to a second, medium shear rate sufficient to form a vortex; e.g. in the range of 65-98 rpm, and about ⅔ of the total methylcellulose may be added in several increments to the mixture. For example, if ⅔ of the total methylcellulose equals 50 gms, it may be added to the mixture in increments of 15 gins, 15 gms and 20 gms. The resulting mixture may then be mixed until a foam of microbubbles appears on the surface layer.

The shear rate of the mixing device may then be increased to a third, high shear rate; e.g. in the range of 98-130 rpm, and the remaining amount of methylcellulose may be added until all of the microbubbles are incorporated into the base material. The added liquid osmotically passes from the base material and into the microbubbles as they are formed and dispersed to provide the desired microencapsulations. The resultant product is a white, fully bonded hydrogel comprising a plurality of microencapsulations that are encapsulated in the base material of plasticized hydrocarbon gel and methylcellulose. The shear rate may then be reduced to a low shear rate; e.g. in the range of 45-65 rpm, and mixing is continued for 2-5 minutes. The hydrogel may be allowed to sit for 1-78 hours before being placed in a suitable container so as to let the microencapsulations settle and reduce in size, thereby increasing the number of encapsulations per volume.

In another embodiment of a process for preparing the ointment of the invention, PEG comprising a mixture of heavy hydrocarbon waxes having a molecular weight of 1295-1315 may be heated until all the waxes have liquefied, typically at a temperature above 95° F. (35° C.). A mixture of mineral oils (%50% light/50% heavy) may be added to the heated PEG, brought to the same temperature to maintain the PEG and oils in liquid form, and continuously mixed in an homogenizer at a low shear rate, e.g. 45-65 rpm. With continued mixing, the mixture may then be allowed to cool to a temperature below 35° C. to form a plasticized hydrocarbon gel. Separately, an aqueous solution, e.g. a saline solution of sodium chloride and water, may be prepared, then any of the liquid or water soluble ingredients (e.g. lavender oil, eucalyptus oil, zinc gluconate, etc.) to be incorporated within the microencapsulations may be mixed into the saline solution, and finally, benzyl alcohol may be added to provide a bacteriostatic saline solution. This solution may then be added to the plasticized hydrocarbon gel while continuing to homogenize the mixture at low shear rate and a temperature below 35° C. Once the mixture is fully mixed, methylcellulose may be added in small amounts until about ⅓ of the methylcellulose is remaining. The mixing speed of the homogenizer may then be increased to a high shear rate, e.g. in the range of 98-130 rpm, until a foam of microbubbles appears, at which point the remaining ⅓ of the methylcellulose may be added. The resulting mixture may then be mixed at the high shear rate until all of the microbubbles of the foam are incorporated and a fully bonded hydrogel is formed. The mixing speed of the homogenizer may then be reduced to the low shear rate, and mixing may be continued until the temperature of the mixture drops to room temperature and a white hydrogel of highly microencapsulated bacteriostatic saline solution and air is formed. Preferably, the hydrogel may be allowed to sit for 1-78 hours so that the microencapsulations can settle and reduce in size, thereby increasing the number of microencapsulations per volume. Then the hydrogel may be placed in suitable containers, e.g. bottles, which may then be refrigerated for 2 hours to 4 days to allow the product to thicken to the desired viscosity. The temperature of the bottles may then be returned to room temperature and the product will remain relatively stable.

The rate at which the base material is mixed or whipped is related to the size of the microencapsulations created within the base material. As mentioned above, the faster and shorter the mixing process, the larger the encapsulations will be. The size of the encapsulations is relevant because this is related to the rate at which the aqueous solution and the additional substances are delivered to the body area to which the ointment is applied. For example, the ointment may be produced with encapsulations having a mean size in the range from about 0.01 mm to about 3 mm. The rate of melting may vary depending upon the temperature of the tissue and its location. For example, when placed in the nasal cavity, the user's breathing convects cooler air over the ointment to reduce its melting rate.

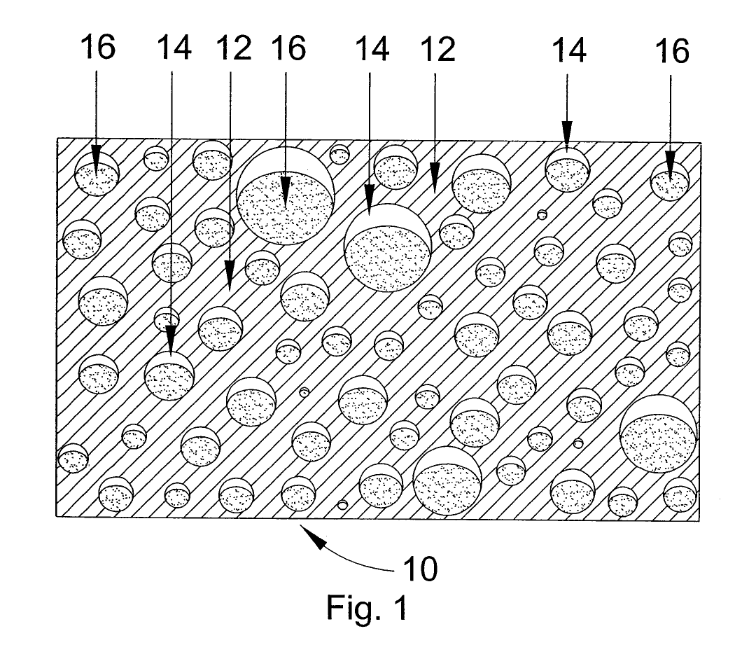

One example of an ointment 10 that may be produced using the process of present invention is illustrated in FIG. 1. Ointment 10 may include a base material 12 containing a plurality of microencapsulations 14 that are encapsulated within base material 12. Within microencapsulations 14 is an amount of a liquid 16. Liquid 16 may partially or completely fill each microencapsulation 14.

If additional substances are to be added, they are added to the liquid, e.g. the bacteriostatic saline solution, before mixing the liquid with the base material. If loss of the additional substance may be a problem (i.e., due to splashing, etc.) the additional substance may be added to the liquid in smaller portions. It should be noted that the above process may be performed at room temperature. However, the ambient temperature should not exceed the melting temperature of the base material.

The percentages of the base material, aqueous solution, and additional substances used during the formulation process can be varied depending upon the type of additional material employed and the desired rate of delivery of the encapsulated materials. For example, if the percentage of base material versus aqueous solution and additional substances is increased, the delivery rate will be decreased. Conversely, if the percentage is decreased, the rate of delivery will increase. As an example, the formulation for the topical ointment may comprise approximately 10-80% by weight of the base material. Additionally, the formulation may comprise approximately 0-90% by weight of the aqueous solution. In formulations where an additional substance is included, 0-40% by weight of the additional substance may be included.

In use, the ointment can be applied directly to various body surfaces. The base material may have a melting temperature of approximately 90° Fahrenheit. Upon application to a body surface, the body heat, which may be approximately 90° to 105° Fahrenheit, will begin to dissolve the bonds of the polymer, e.g. polyethylene glycol, in the base material, which in turn releases an amount of the encapsulated aqueous solution and additional substance, if encapsulated, to the body area. The combination of the aqueous solution and mineral oil in the base material acts as a moisturizing element for the exposed area.

In a preferred embodiment using a plasticized hydrocarbon gel comprising mineral oil and PEG, the slow release of the aqueous solution and additional substances is made possible by the fact that, due to its chemical make-up, the PEG will retain its structure, bond the mineral oil and hold the encapsulated material until a melting temperature is reached. When an amount of the ointment is applied to a body area, the portion of the ointment in contact with the skin will have a higher temperature than the amount which is further from the skin. In practice, the portion of the ointment next to the skin will dissolve, delivering its encapsulated material to the area, while the portions away from the skin will remain cooler and thus retain the encapsulated materials. In some embodiments, a topical application of the ointment of the present invention may last up to 8 hours and sometimes as much as 12 hours.

For example, the ointment of the present invention has special applicability for use within the nasal cavity upon the nasal mucosa. Upon application, the portion in contact with the skin will dissolve and deliver the encapsulated substances. Conversely, the portion of the ointment exposed to the air will stay in the gelatinous state due to, in most cases, the lower ambient temperature of the air. The cooler temperatures are also due to evaporative cooling effects which are caused by the movement of air through the nasal cavity. This provides the benefit that the entire amount of ointment applied to the bodily surface does not dissolve and expose the entire amount of the encapsulated material to the area at once. All of the dissolved materials may be absorbed by the skin and the delivery of the additional substance to the bloodstream may occur. The ointment may be applied to other mucosal surfaces (vagina, rectum) but the rate of delivery may be greater due to the lack of moving air. The ointment may also be applied to any patches of dry skin in need of moisture.

### EXAMPLE 1

Ingredients Used:

908 gms of plasticized hydrocarbon gel base [95% mineral oil (50% light and 50% heavy) and 5% heavy hydrocarbon waxes (polyethylene glycol) having a molecular weight of 1295-1315] (Plastibase® from Bristol-Meyers Squibb). 820 mL of 0.9% Sodium Chloride solution

6.3 mL of Benzyl Alcohol

55 gms of Methylcellulose 4000 cps

All 908 grams of solid bulk plasticized hydrocarbon gel may be placed in a mixing bowl of a mixing apparatus, such as a table top mixer. The mixer may be started at a low shear rate and the plasticized hydrocarbon gel may be mixed for 2-8 minutes. Then, the 820 mL of sodium chloride solution may be added in increments of 200-250 mL over a period of 2-5 minutes until all of the solution has been added and mixed with the plasticized hydrocarbon gel. Next, the benzyl alcohol may be added in 1-1.5 mL increments until all 6.3 mL have been added. The combination of sodium chloride solution and benzyl alcohol forms a bacteriostatic saline solution mixed in with the plasticized hydrocarbon gel.

The shear rate of the mixing apparatus may then be increased to medium for 1-3 minutes. Once a vortex is formed, 35 gms of the methylcellulose may be added in 5-10 gm increments. The medium shear rate may be continued until a foam of microbubbles appears on the surface layer, which indicates the complexation of the polyethylene glycol (PEG) and methylcellulose to form a hydrogel. The shear rate may then be increased to high and the remaining 20 gms of methylcellulose may be added and mixed until all of the foam become part of the base material. The resultant product is a white, fully bonded hydrogel comprising highly microencapsulated bacteriostatic saline solution and air. Shear rate of the mixing apparatus may then be reduced to low for 2-5 minutes. The resulting composition may be allowed to sit for 1-78 hours for the microencapsulations to settle and reduce in size (thereby increasing the encapsulations per volume). The composition may then be used immediately or packaged in a suitable container for future use. If the ointment is to be applied in the nasal cavity, a typical dosage may be 0.1 g, or 100 mg, per nostril. Upon application, the total amount may dissolve in approximately a 8-12 hour period.

### EXAMPLE 2

Ingredients Used:

862.6 gms of a mixture of mineral oils (50% light and 50% heavy)

45.4 gms of polyethylene glycol (PEG) having a molecular weight of 1295-1315

820 mL of 0.9% Sodium Chloride solution

6.3 mL of Benzyl Alcohol

55 gms of Methylcellulose 4000 cps

The PEG may be placed in a mixing bowl of an homogenizer and heated until all of the PEG is liquefied. The mineral oil may be added to the heated PEG and brought to the same temperature to maintain the mixture in liquid form. The mixture may then be continuously mixed at a low shear rate and then allowed to cool to a temperature below 35° C. Separately, the benzyl alcohol may be added to the sodium chloride solution to form a bacteriostatic saline solution. This solution may then be added to the PEG/mineral oil mixture while continuing to homogenize the mixture at a low shear rate. Once fully mixed, about 37 gms of the methylcellulose may be added in small increments to the mixture. The speed of the homogenizer may then be increased to a high shear rate until a foam appears, at which point the remaining 18 gms of methylcellulose may be added. The mixture may then be mixed until all of the microbubbles of the foam are incorporated and a fully bonded hydrogel is formed. The mixing speed of the homogenizer may then be reduced to a low shear rate and the mixture may continue to be mixed until the temperature of the mixture drops to room temperature and a white hydrogel with highly microencapsulated bacteriostatic saline solution is formed. The resulting composition may be allowed to sit for 1-78 hours for the microencapsulations to settle and reduce in size (thereby increasing the encapsulations per volume). The composition may then be packaged in a bottle and refrigerated for from 2 hours to 4 days to allow the product to thicken to the desired viscosity. The bottles can then be returned to room temperature and the product will remain relatively stable.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant are, within the scope of the present invention. The embodiments described hereinabove are further intended to explain best modes known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed is:

1. A topical ointment comprising:
a base material comprising plasticized hydrocarbon gel and methylcellulose; and
a plurality of microbubbles containing liquid,
wherein the microbubbles are encapsulated by the base material to form microencapsulations, and the microencapsulations are dispersed in the base material to form a hydrogel.

2. The topical ointment of claim 1 wherein the base material is configured to melt when placed on living tissue that is at a temperature in the range from about 90° F. to about 105° F.

3. The topical ointment of claim 1 wherein the base material comprises about 90-97% plasticized hydrocarbon gel and about 3-10% methycellulose.

4. The topical ointment of claim 1 wherein the plasticized hydrocarbon gel comprises 91-99% mineral oil and 1-9% polyethylene glycol.

5. The topical ointment of claim 1 wherein the methylcellulose has a viscosity of 3000-5600 cps.

6. The topical ointment of claim 1 wherein the liquid is water or an aqueous solution.

7. The topical ointment of claim 6 wherein the aqueous solution comprises a bacteriostatic combination of a 0.9% saline solution and benzyl alcohol.

8. The topical ointment of claim 1 further comprising one or more additives selected from the group consisting of antibiotics, steroids, aromatic oils, dietary supplements, humalog insulin, nitro-glycerin, anti-nausea medications, pain killers and smoking cessation medications.

9. The topical ointment of claim 8 wherein the additive comprises Lavender Oil, Eucalyptus Oil or zinc gluconate.

10. The topical ointment of claim 1, wherein the microencapsulations have a mean size in the range from about 10 microns to about 1500 microns.

11. The topical ointment of claim 1 comprising 10-80% by weight of the base material and 0-90% by weight of liquid, and optionally 0-40% of an one or more additives selected from the group consisting of antibiotics, steroids, aromatic oils, dietary supplements, humalog insulin, nitro-glycerin, anti-nausea medications, pain killers and smoking cessation medications.

12. A method of preparing a topical ointment comprising the steps of:
providing a plasticized hydrocarbon gel;
mixing the plasticized hydrocarbon gel with a liquid solution;
mixing a first quantity of methylcellulose into the mixture in small incremental amounts;
increasing the mixing shear rate to a level sufficient to form microbubbles on the surface;
adding a second quantity of methylcellulose into the mixture and continuing mixing at the higher shear rate until the microbubbles are incorporated into the mixture and the liquid has moved into the microbubbles to form microencapsulations; and
reducing the mixing shear rate and continuing to mix the mixture for a time sufficient to fully disperse the microencapulations and form a fully bonded hydrogel.

13. The method of claim 12, wherein the hydrogel is allowed to sit for 1-78 hours so as to let the microencapsulations settle and reduce in size.

14. The method of claim 12, further comprising adding at least one additional substance to the liquid and mixing the base material and liquid until the liquid and the at least one additional substance are contained within the microbubbles.

15. The method of claim 12 wherein the plasticized hydrocarbon gel comprises 91-98% mineral oil and 1-9% polyethylene glycol.

16. A method of preparing a topical ointment comprising the steps of:
mixing a plasticized hydrocarbon gel with a liquid at a first low shear rate sufficient to provide a gel/liquid mixture;
mixing the gel/liquid mixture at a second shear rate sufficient to form a vortex;
adding methylcellulose to the vortex in several increments until about ⅔ of the methyl cellulose has been added, and continuing to mix the gel/liquid/methylcellulose mixture at the second shear rate until a foam of microbubbles appears on the surface layer;
mixing the gel/liquid/methycellulose mixture at a third higher shear rate sufficient to form microbubbles of the desired size while adding the remaining methylcellulose;
continuing to mix the gel/liquid/methycellulose mixture for a time sufficient to incorporate all of the microbubbles into the mixture and allow the liquid to move into the microbubbles; and
mixing the gel/liquid/methylcellulose mixture at the first shear rate for a period of time sufficient to fully disperse the microencapsulations in the mixture and form a fully bonded hydrogel.

17. The method of claim 16 wherein the first shear rate is in the range of 45-65 rpm, the second shear rate is in the range of 65-98 rpm, and the third shear rate is in the range of 98-130 rpm.

18. A method of preparing a topical ointment comprising the steps of:
heating a quantity of polyethylene glycol until it is liquefied;
mixing mineral oil with the heated liquefied polyethylene glycol at a low shear rate;
while continuing mixing at the low shear rate, (a) cooling the mixture to a temperature below 35° C., (b) adding a liquid solution thereto, and (c) adding methylcellulose in several increments until about ⅔ of the methyl cellulose has been added;
mixing the resultant mixture at a high shear rate sufficient to form microbubbles on the surface;
adding the remaining amount of methylcellulose;
continuing to mix the resultant mixture for a time sufficient to incorporate all of the microbubbles into the mixture and allow the liquid to move into the microbubbles to form microencapsulations; and
mixing the resultant mixture at a low shear rate for a period of time sufficient to reduce the temperature of the mixture to room temperature and fully disperse the microbubbles in the mixture and form a fully bonded hydrogel.

19. A method for delivering a substance to living tissue, the method comprising:
providing an ointment wherein a plurality of microbubbles containing liquid are encapsulated by a base material comprising plasticized hydrocarbon gel and methycellulose to form a hydrogel of microencapsulations dispersed in the base material, and wherein the base material is configured to melt when placed on living tissue that is at a temperature in the range from about 90° F. to about 105° F., and
applying the ointment to living tissue to permit the base material to melt over time due to the heat of the living tissue and to slowly release the liquid to the living tissue.

20. The method of claim 17, wherein the living tissue comprises the nasal mucosa.

21. The method of claim 17 wherein the microencapsulations have a mean size in the range from about 10 microns to about 1500 microns.

22. The method of claim 17, wherein the liquid comprises water or an aqueous solution.

23. The method of claim 20, wherein the liquid comprises a bacteriostatic saline solution comprising sodium chloride, benzyl alcohol and sterile H₂O.

24. The method of claim 17, wherein the ointment further includes one or more additional substances selected from a group consisting of antibiotics, steroids, aromatic oils, dietary supplements, humalog insulin, nitro-glycerin, anti-nausea medications, pain killers and smoking cessation medications, and wherein the additional substance is included within the gas microbubbles and is released to the living tissue as the base material melts.

25. The method of claim 17 wherein the topical ointment comprises 10-80% by weight of the base material and 0-90% by weight of liquid.

26. The method of claim 18, wherein the base material comprises about 90-97% plasticized hydrocarbon gel and about 3-10% methycellulose, and the plasticized hydrocarbon gel comprises 91-99% mineral oil and 1-9% polyethylene glycol.
